(19) United States
(12) Patent Application Publication
(10) Pub. No.: US 2020/0273562 A1
(43) Pub. Date: Aug. 27, 2020

(54) AUTOMATED HEALTHCARE STAFFING SYSTEM

(71) Applicant: LPD2, Limited, Henderson, NV (US)

(72) Inventor: Dante Tolbert, Henderson, NV (US)

(21) Appl. No.: 16/790,654

(22) Filed: Feb. 13, 2020

Related U.S. Application Data
(60) Provisional application No. 62/808,460, filed on Feb. 21, 2019.

(51) Int. Cl. G16H 40/20; G06Q 10/06

(52) U.S. Cl. CPC G16H 40/20; G06Q 10/063118

(57) ABSTRACT

Automated healthcare staffing systems and methods can include receiving statistical data from multiple data sources, calculating numerical values for data metrics, assigning confidence multipliers to the metrics, using the numerical values and confidence multipliers in a statistical model to compute a predicted patient census, generating and displaying a graphical representation of the predicted patient census, providing information regarding the number of nurses scheduled to work a future shift and the number of nurses that are needed for the shift based on the predicted patient census, assigning internal nurses to work the future shift if the need exceeds the number scheduled and internal nurses are available, and sending an alert to nurses employed by an outside staffing agency if the need exceeds the number scheduled and internal nurses are not available.

### CROSS-REFERENCE TO RELATED APPLICATION

This application claims the benefit of U.S. Provisional Patent Application No. 62/808,460 filed on Feb. 21, 2019 and entitled “SYSTEM FOR PREDICTING HEALTHCARE LABOR EFFECTIVENESS INFORMATION AND LABOR PROCUREMENT,” which application is hereby incorporated by reference in its entirety.

### TECHNICAL FIELD

The present disclosure relates generally to workplace staffing, and more particularly to the staffing of professional workers in healthcare facilities.

### BACKGROUND

Staffing professionals for hospitals, clinics, and other healthcare organizations are typically tasked with anticipating patient numbers and needs and then matching appropriate levels of nurses and other healthcare staff to meet these needs in the scheduling process. While specific productivity measures and goals vary across organizations and treatment settings, common aims are to measure and improve on labor costs per units of service, such as the numbers of occupied beds, adjusted occupied beds, patient visits, clinical interventions, and the like. Relevant data for these items often resides across multiple disparate systems or is manually tabulated on physical bed boards, leading to suboptimal outcomes in decision making. In many instances, staffers simply guess at the amounts of nurses that will be needed based on experience and intuition, and then manually set nurse staffing schedules accordingly.

Although traditional ways of staffing healthcare professionals have worked well in the past, improvements are always helpful. In particular, what is desired are healthcare staffing systems and methods that monitor, anticipate, and respond to changes in patient data across healthcare facilities in order to make efficient and effective decisions for the scheduling and staffing of healthcare professionals.

### SUMMARY

It is an advantage of the present disclosure to provide automated healthcare staffing systems that efficiently schedule and staff nurses and other healthcare professionals for hospitals, clinics, and other healthcare facilities. The disclosed features, apparatuses, systems, and methods provide improved staffing solutions that involve accurately forecasting patient censuses in order to schedule nurses and other healthcare professionals in a more efficient manner.

These advantages can be accomplished in multiple ways, such as by collecting statistical data from multiple disparate data sources and then analyzing the collected data to arrive at predicted patient censuses at the healthcare facilities for some or all future dates. A user can interact with the system to view historical data, predicted patient censuses, and future nurse scheduling, such that action can be taken to adjust the number of nurses scheduled for upcoming shifts, if needed.

In various embodiments of the present disclosure, methods for staffing a healthcare facility can be performed by a computer system that includes a user interface element configured to receive user input. These methods can include storing a set of nurse to patient ratio relationships on the computer system, receiving at the computer system statistical data from a plurality of data sources, calculating numerical values for a plurality of metrics based on items within the statistical data, wherein each of the plurality of metrics relate to a patient census within the healthcare facility, assigning confidence multipliers to each of the metrics having a numerical value, wherein each confidence multiplier ranges from 0 to 1, using the numerical values and confidence multipliers in at least one statistical model to compute a plurality of outputs, saving the outputs on the computer system, generating a graphical representation of the outputs, displaying the graphical representation of the outputs to a user of the computer system, accepting at the computer system a request from the user to analyze a selected unit of the healthcare facility, providing information to the user regarding the number of nurses scheduled to work a future shift at the selected unit and the number of nurses that are needed to work the future shift based on a predicted patient census for the selected unit, determining whether the number of nurses scheduled is greater than or equal to the number of nurses that are needed to work the future shift at the selected unit, determining whether any internal nurses are available when the number of nurses that are needed is greater than the number of nurses that are scheduled to work the future shift at the selected unit, assigning one or more internal nurses to work the future shift at the selected unit when the number of nurses that are needed is greater than the number of nurses that are scheduled to work the future shift at the selected unit and internal nurses are available, and sending an alert to nurses employed by an outside staffing agency when the number of nurses that are needed is greater than the number of nurses that are scheduled to work the future shift at the selected unit and internal nurses are not available. Each of the nurse to patient ratio relationships can reflect the maximum number of patients permitted per nurse for a unit of the healthcare facility. The plurality of data sources can include at least a scheduling system, an admission discharge transfer system, and an electronic medical record system. Each output can relate to a predicted patient census for a unit of the healthcare facility, and the outputs can include at least a predicted patient census for an emergency department, a predicted number of patients admitted as inpatient to the healthcare facility from the emergency department, and a predicted number of inpatient discharges from the healthcare facility. Internal nurses can be nurses employed by the healthcare facility.

In various detailed embodiments, methods can include providing to the user a list of available nurses when the number of nurses that are needed is greater than the number of nurses that are scheduled to work the future shift at the selected unit, and the list of available nurses can include internal nurses and nurses employed by an external staffing agency. Methods can also include converting a predicted patient census for the selected unit to the number of nurses that are needed to work the future shift based on a stored nurse to patient ratio relationship for the selected unit, as well as dynamically updating the predicted patient census for the selected unit as the future shift approaches. The at least one statistical model can include a seasonal autoregressive integrated moving average statistical model, and the plurality of data sources can also include a weather application, a healthcare news application, or an infectious disease tracker. The graphical representation can include a combination of historical and forecasted patient census information, and information provided to the user can include nurses currently scheduled for the future shift and nurses who have called out of the future shift. The methods can also include calculating a predicted nurse census for the future shift at the selected unit and increasing the number of nurses scheduled to work the future shift when the number of nurses that are needed is greater than the predicted nurse census.

In various other embodiments of the present disclosure a non-transitory computer-readable medium can contain instructions for staffing a healthcare facility that include some or all of the foregoing method steps. In addition, a system configured for automated healthcare facility staffing can include at least one memory and a processor coupled thereto and configured to execute processor-executable instructions contained in the at least one memory. The instructions can be for staffing a healthcare facility and again can include instructions for some or all of the foregoing method steps.

Other apparatuses, methods, features, and advantages of the disclosure will be or will become apparent to one with skill in the art upon examination of the following figures and detailed description. It is intended that all such additional apparatuses, methods, features and advantages be included within this description, be within the scope of the disclosure, and be protected by the accompanying claims.

### BRIEF DESCRIPTION OF THE DRAWINGS

The included drawings are for illustrative purposes and serve only to provide examples of possible structures and arrangements for the disclosed apparatuses, systems and methods for automated healthcare staffing These drawings in no way limit any changes in form and detail that may be made to the disclosure by one skilled in the art without departing from the spirit and scope of the disclosure.

FIG. 1A illustrates a diagram of an example environment in which various embodiments disclosed herein can operate according to one embodiment of the present disclosure.

FIG. 1B illustrates a diagram of an example computer system that can execute instructions to perform various methods disclosed herein according to one embodiment of the present disclosure.

FIG. 1C illustrates a diagram of an example automated healthcare staffing system with functional callouts between items according to one embodiment of the present disclosure.

FIG. 2A illustrates a flowchart of an example method of providing a predicted patient census for a healthcare facility according to one embodiment of the present disclosure.

FIG. 2B illustrates a flowchart of a more detailed example method of providing a predicted patient census for a healthcare facility according to one embodiment of the present disclosure.

FIG. 3A illustrates an example screenshot of information for an automated healthcare staffing system according to one embodiment of the present disclosure.

FIG. 3B illustrates an example graphical user interface for an automated healthcare staffing system according to one embodiment of the present disclosure.

FIG. 3C illustrates an example graphical user interface for internal staff of an automated healthcare staffing system according to one embodiment of the present disclosure.

FIG. 3D illustrates another example graphical user interface for internal staff of an automated healthcare staffing system according to one embodiment of the present disclosure.

FIG. 3E illustrates an example graphical user interface for external staff of an automated healthcare staffing system according to one embodiment of the present disclosure.

FIG. 3F illustrates another example graphical user interface for external staff of an automated healthcare staffing system according to one embodiment of the present disclosure.

FIG. 4 illustrates a flowchart of example details for analyzing statistical data in order to provide staffing for a healthcare facility according to one embodiment of the present disclosure.

FIG. 5 illustrates a flowchart of an example method of providing automated staffing for a healthcare organization according to one embodiment of the present disclosure.

FIG. 6 illustrates a diagram of an example computer that may perform processing for various embodiments and components of the present disclosure.

### DETAILED DESCRIPTION

Exemplary applications of apparatuses, systems, and methods according to the present disclosure are described in this section. These examples are being provided solely to add context and aid in the understanding of the disclosure. It will thus be apparent to one skilled in the art that the present disclosure may be practiced without some or all of these specific details provided herein. In some instances, well known process steps have not been described in detail in order to avoid unnecessarily obscuring the present disclosure. Other applications are possible, such that the following examples should not be taken as limiting. In the following detailed description, references are made to the accompanying drawings, which form a part of the description and in which are shown, by way of illustration, specific embodiments of the present disclosure. Although these embodiments are described in sufficient detail to enable one skilled in the art to practice the disclosure, it is understood that these examples are not limiting, such that other embodiments may be used, and changes may be made without departing from the spirit and scope of the disclosure.

In addition, it will be understood that steps of the exemplary methods set forth herein can be performed in different orders than the order presented. Furthermore, some steps of the exemplary methods may be performed in parallel rather than being performed sequentially. Also, the steps of the exemplary methods may be performed in a network environment in which some steps are performed by different computers in the networked environment.

Various embodiments of the present disclosure relate to the staffing of professional workers in healthcare organizations. In particular, some embodiments relate to the automated forecasting of predicted patient censuses at healthcare facilities for one or more future dates. The disclosed embodiments can provide their advantages to many kinds of healthcare facilities, including hospitals, clinics, urgent cares, doctor offices, and the like. Although staffing professionals at these facilities may simply use the provided predicted patient censuses to construct staffing schedules manually, the various systems and methods disclosed herein may include further automated features that act upon the predicted patient censuses to provide additional advantages.

The disclosed systems and methods automatically match clinical labor to patient demand by aggregating operational data from many disparate systems and utilizing standard statistical methods and machine learning to forecast both patient demand and clinical labor supply. Various embodiments may operate in conjunction with existing staff schedules to compare future shift schedules with the number of staff needed to accommodate predicted patient censuses that correspond with the future shift schedules. The disclosed systems and methods also facilitate procuring qualified and available contingent labor manually and/or automatically when the number of staff scheduled for a shift is inadequate to meet the forecasted need. User interfaces allow for scheduling staff, automated load balancing clinical labor in Some embodiments may be a component or module of an overall system or program, such as a data analysis component or a staff procurement component or module.

Various embodiments disclosed herein can include system displays and interfaces that provide some or all of the following features:
a current accounting of all assigned clinical staff by date,
a current accounting for all previously assigned staff that are no longer scheduled,
available clinical staff employed by the healthcare organization and their associated costs,
available external contingent staff meeting the clinical and professional inclusion criteria set by the healthcare organization and their associated costs,
past staffing levels and associated patient census levels,
forecasted patient demands,
forecasted employee deficiencies, and
forecasted clinical staff supply.
   Each of these items can be provided live and be dynamically updated, and forecasted items can be calculated continuously by both standard statistical methods and machine learning methods.

Although the disclosed embodiments refer primarily to nurses as the specific type of healthcare personnel being staffed, it will be understood that other personnel might also be staffed by way of the disclosed systems and methods. For example, doctors, surgeons, technicians, desk personnel, security, and other personnel may also be staffed by the same or similar systems and methods. In addition, while the disclosed embodiments refer to hospitals and other healthcare organizations, it will be readily appreciated that these systems and methods may also be used to provide workplace staffing for may other disciplines and industries.

### Example Environments

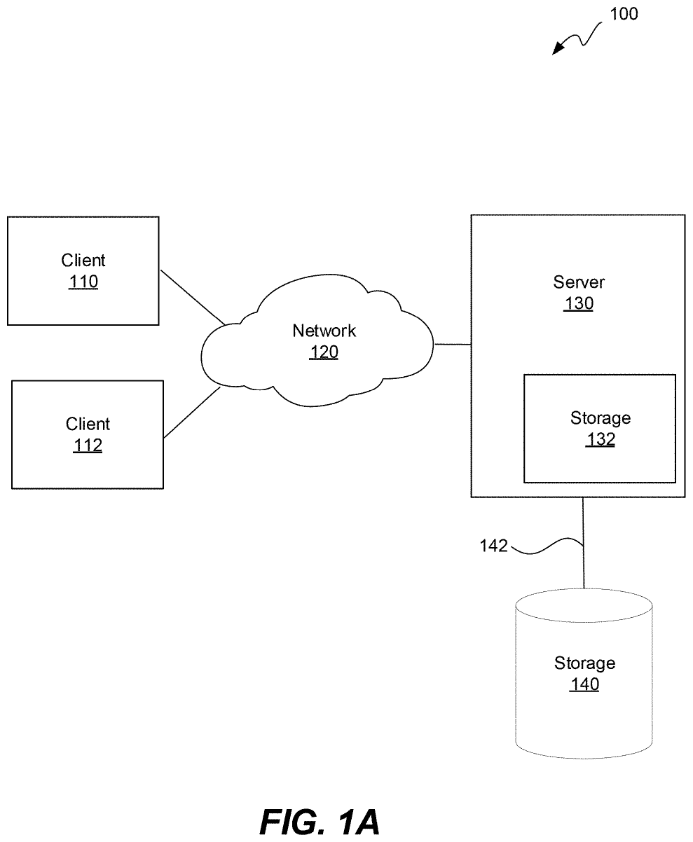

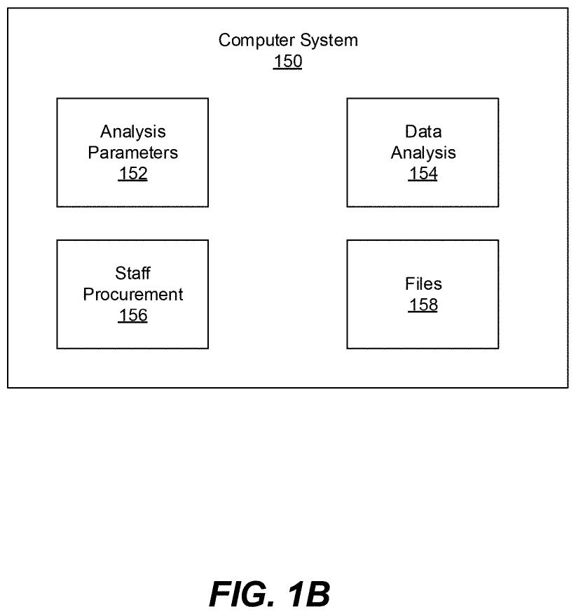

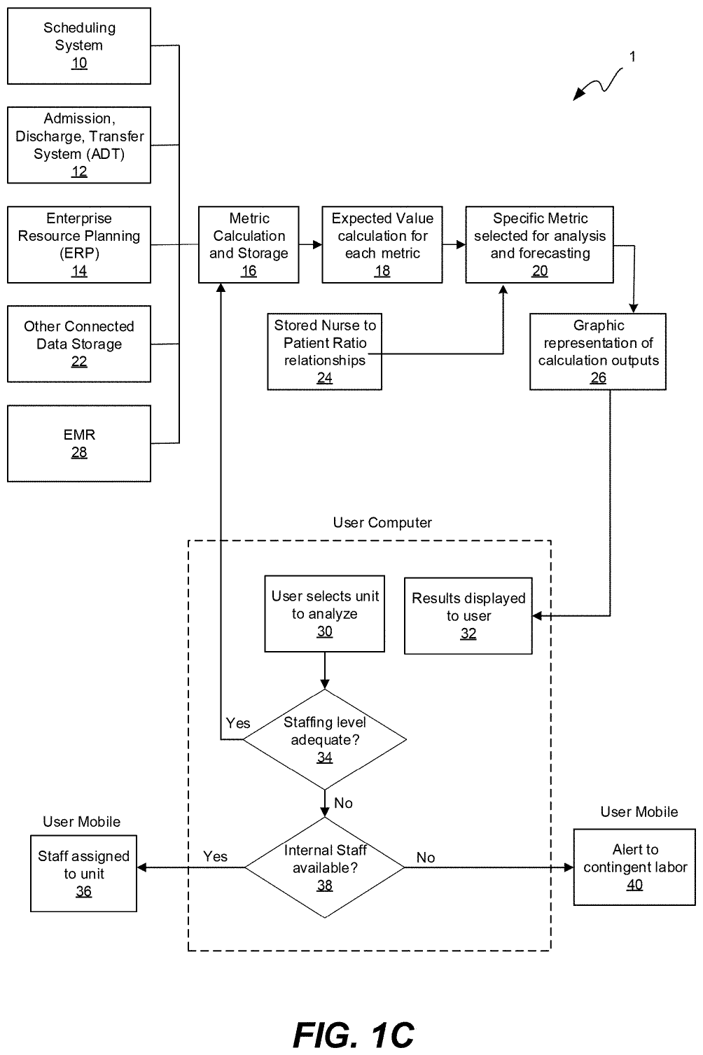

Starting with FIG. 1A, a diagram is provided of an example environment in which various embodiments disclosed herein can operate. System 100 can include two clients 110 and 112 that are connected over a network 120 to a server 130 having a local storage 132. Clients and servers in this environment may be computers or computer systems. Server 130 may be configured to handle requests from clients. Although shown as a single entity, server 130 may be implemented as multiple distributed and networked server devices. While system 100 is illustrated with only two clients and one server for purposes of simplicity, it will be understood that there may be more or fewer clients and servers. The computers have been termed clients and servers, although clients can also play the role of servers and servers can also play the role of clients. In some embodiments, the clients 110 and 112 may communicate with each other as well as the server(s). Also, the server 130 may communicate with other servers.

The network 120 may be, for example, a local area network (“LAN”), a wide area network (“WAN”), telephone networks, wireless networks, intranets, the Internet, or combinations of networks. The server 130 may be connected to an external storage 140 over a connection medium 142, which may be a bus, crossbar, network, or other interconnect. Storage 140 may be implemented as a network of multiple storage devices, though it is illustrated as a single entity. Storage 140 may be a file system, disk, database, or other storage.

Continuing with FIG. 1B, another diagram depicts an example computer system that can execute instructions to perform various methods disclosed herein. Computer system 150 may include different components or software modules configured to execute some of the functions disclosed herein. It will be understood that the components or modules shown here are for purposes of illustration, and that these components or modules may be separated into further subcomponents or submodules. Also, one or more additional components or modules may be added.

Analysis Parameters 152 may provide stored preferences, values, formulas, and other items used to forecast future patient censuses for a healthcare facility. These may include, for example, numerical values for different data categories, confidence multipliers to assign to various numerical values based on different situations, stored nurse to patient ratio relationships (item 24 of FIG. 1C), and preferred formulas to use for different units and situations, among other analysis parameters.

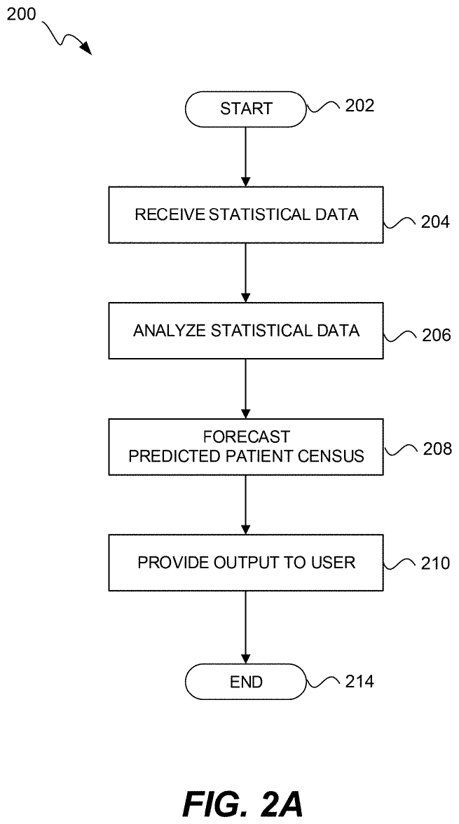

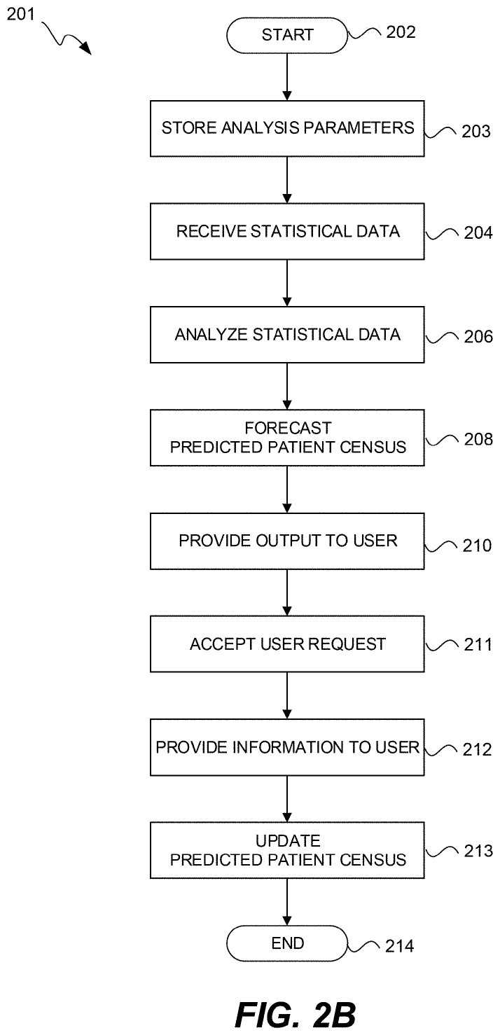

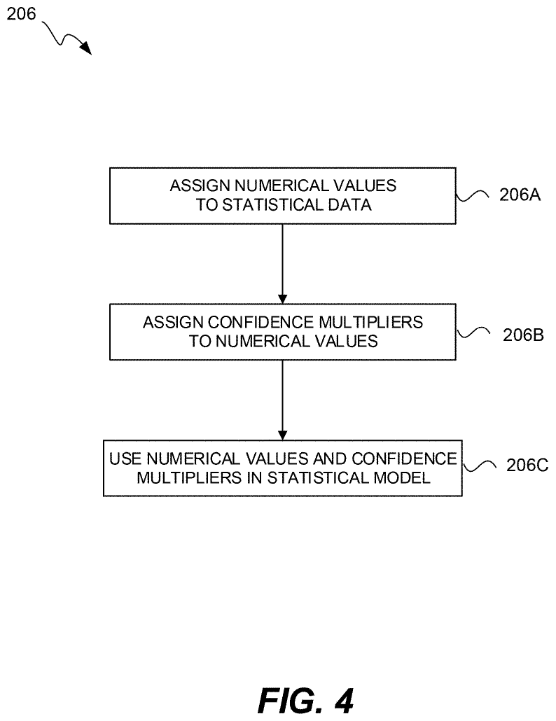

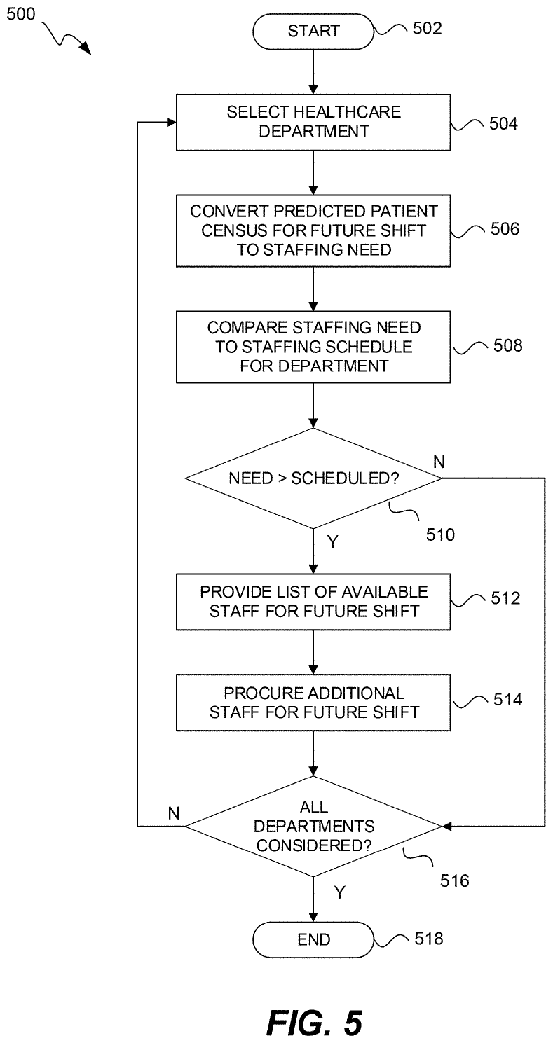

Data Analysis 154 may perform forecasting predicted patient census functionality, which may include acquiring data from various disparate data sources and applying the various values and parameters stored in the Analysis Parameters 152 to arrive at a statistically calculated result and confidence level for a predicted patient census on a given future date. For example, Data Analysis 154 may include metric calculation and storage (item 16 of FIG. 1C), and may perform steps 206, 208, and 213 of FIG. 2B, all steps of FIG. 4, and steps 506 and 508 of FIG. 5, among other possible functions.

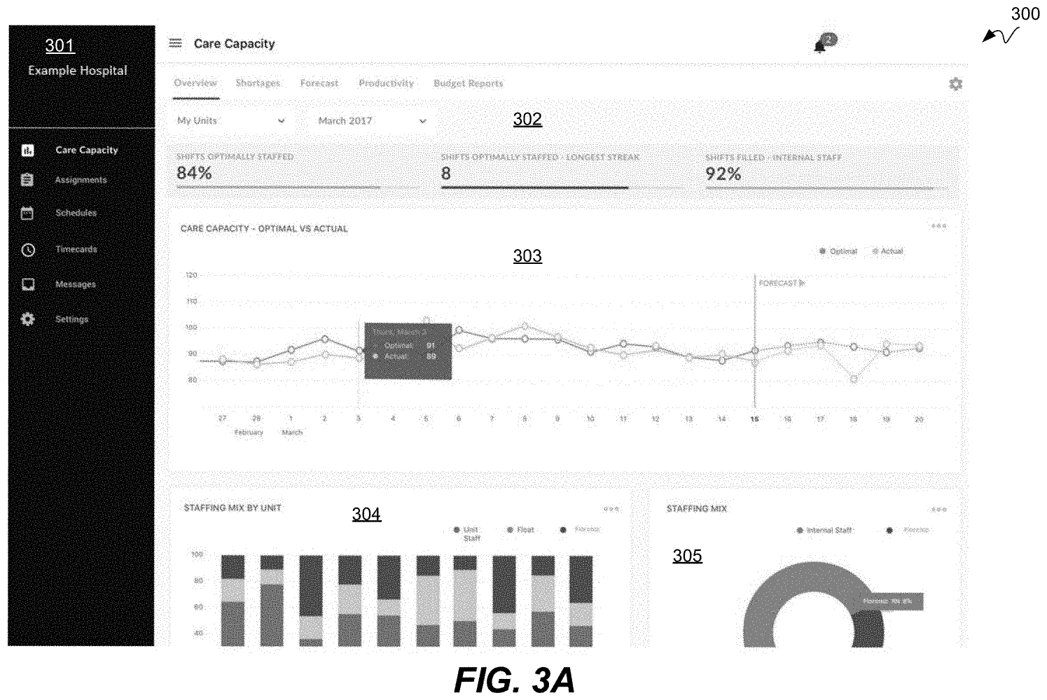

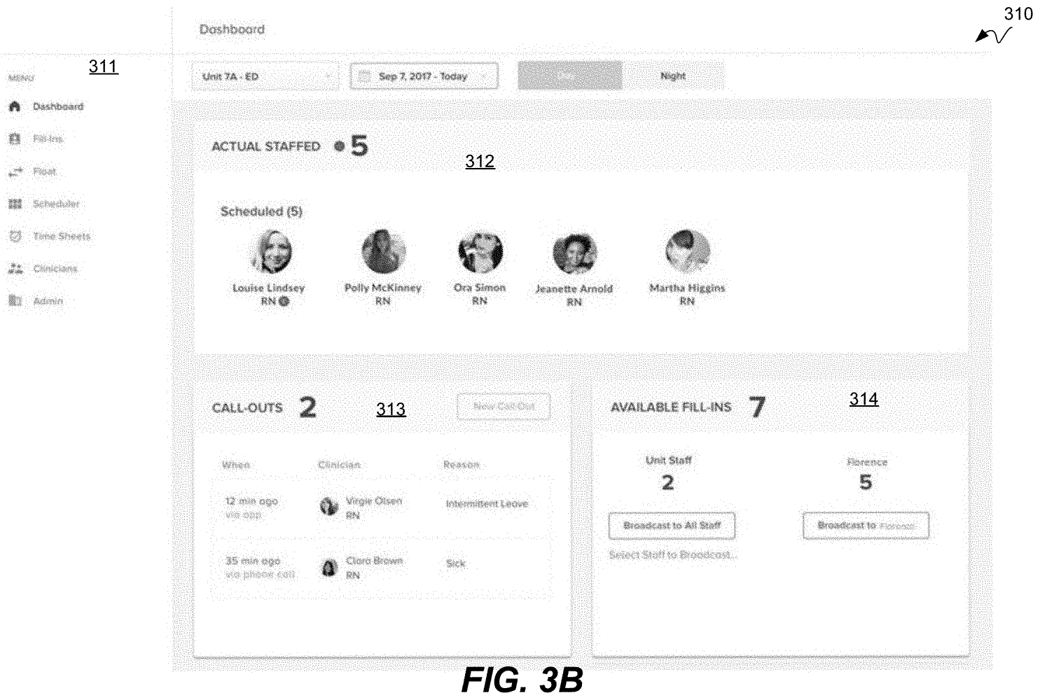

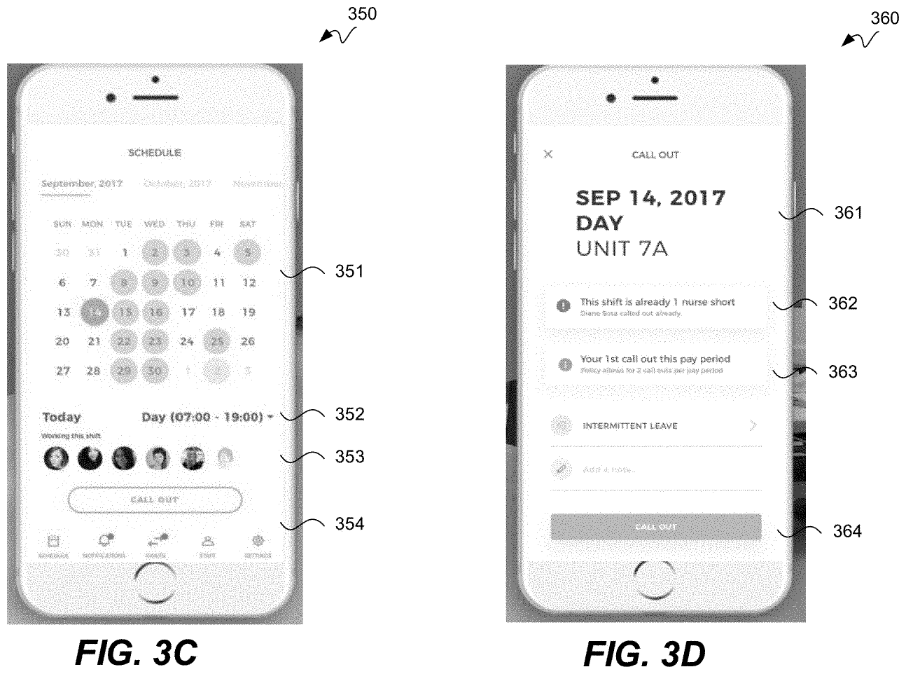

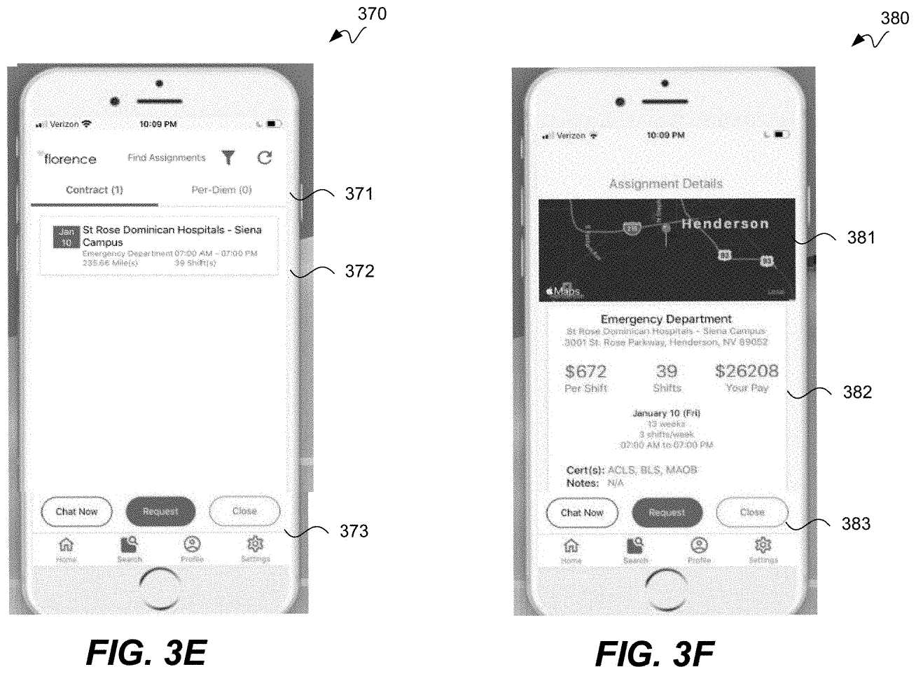

Staff Procurement 156 may perform procuring additional staff functionality, which can include adding extra staff when an upcoming shift is understaffed with respect to its predicted patient census. This can include, for example, providing to a staffer a list of available nurses when the number of nurses needed is greater than the number of nurses scheduled. The list of available nurses can include nurses employed by the healthcare facility and nurses employed by an external staffing agency. Staff Procurement 156 may include, for example, steps 34-40 of FIG. 1C, the various features of FIGS. 3B-3D, and steps 508-514 of FIG. 5, among other possible items and functions.

Files 158 may include various files and items created, stored, and used by computer system 150 and other associated computer systems within an overall system. Stored files can include those regarding historical patient census and staffing data, as well as predicted patient census files and future staffing schedules, all of which may be updated dynamically. Stored files can also include various data items collected from the various disparate data sources accessed by the system. Files 158 may be accessed by computer system 150, a user thereof, or a system associated computer or user, such as at steps 211 and 212 of FIG. 2B, various displayed items of FIGS. 3A-3F, and step 512 of FIG. 5.

Focusing now on FIG. 1C, a diagram of an example automated healthcare staffing system with functional callouts between items is provided. Automated healthcare staffing system 1 can include a plurality of data sources or data repositories, such as a Scheduling System 10, an Admission, Discharge, Transfer (“ADT”) System 12, an Enterprise Resource Planning (“ERP”) System 14, Other Connected Data Storage 22, and an Electronic Medical Records (“EMR”) System 28, for example. Automated healthcare staffing system 1 can collect pertinent items of statistical data from some or all of these various disparate data sources in order to analyze the statistical data and provide accurate predicted patient censuses for future shifts at different units of an associated healthcare facility.

Scheduling System 10 can include one or more different schedules for a healthcare facility associated with system 1. A patient schedule can include information regarding upcoming procedures and inpatient stays for patients at the healthcare facility. These can include, for example, scheduled non-urgent surgeries such as gallstone removal or hip replacement, as well as scheduled elective surgeries such as bariatric or various cosmetic surgeries. A staff schedule can include information regarding scheduled shifts for healthcare facility staff, such as nurses. Other schedules may also be included in Scheduling System 10, such as a generic calendar noting major holidays. These patient, staff, and other schedules may be part of a single scheduling system or may be separate components of an overall Scheduling System 10 data source category from which the system 1 collects data.

ADT System 12 can include information regarding dates and times of patient admissions, discharges, and transfers. Alerts can also be provided by ADT System 12 when some patients are admitted, such as where a patient has a history of infectious disease or heart ailments. Various items of ADT data can be used to accurately count patient censuses and estimate patterns of patient flow between various units and departments in a hospital or other healthcare facility. These items can include, for example, admit/visit notifications, patient transfers, patient discharges, patient registrations, patient pre-admittances, changes between inpatient or outpatient status, patient information updates, patient departures, patient arrivals, admit/visit cancellations, transfer cancellations, discharge cancellations, pending admissions, pending discharges, pending transfers, patient swaps, patient information merges, bed status updates, patient leaves of absence, and the like.

ERP System 14 can include business management information regarding financials, supply chain, operations, services, reporting, manufacturing, and human resource activities for a healthcare facility. Some of this information can relate to specific patients, such as billing, insurance, accounts receivable, and outstanding balances, while other informational items can relate to the overall management of the healthcare facility. ERP System data can include, for example, unit identifiers, total number of beds, number of occupied beds, dates of occupancy, unit function, numbers of patients by gender, list of patient identifiers, list of bed identifiers, bed statuses, number of nurses by type or specialty, patient to nurse ratios, and the like.

EMR System 28 can include general patient information as well as records of the specific medical and treatment histories for patients at a healthcare facility, which are essentially digital versions of the paper charts used for patients by doctors, nurses, and other medical professionals. Patient data can include, for example, patient name, other identifier(s), date of birth, sex, race, military status, nationality, height, weight, marital status, allergies, medications, immunizations, ambulatory status, address, living arrangement, insurance information, known admissions to healthcare facilities, and the like. Data on known admissions can include, for example, admission dates and times, discharge dates and times, discharge statuses, discharge locations, admission types, admissions sources, diagnoses, procedures, patient outcomes, and the like.

In general, Scheduling System 10, ADT System 12, ERP System 14, and EMR System 28 can be internal data sources that are kept by the healthcare facility itself. Conversely, Other Connected Data Storage 22 can include various external systems having statistical data that may be relevant for forecasting predicted patient censuses at a healthcare facility. These can include, for example, a weather application, a healthcare news source or application, an external events calendar for a region around a healthcare facility, and a flu or other infectious disease tracker for the region, among other external data sources. While some of these external data sources may contain mostly irrelevant data, at least some of their data items can be relevant for predicting patient censuses.

Statistical data from these different internal and external data sources can be compiled, analyzed, and trended at Metric Calculation and Storage 16. Certain portions of the statistical data can be used for different metrics that then form portions of a predicted patient census for a given unit at the healthcare facility. Each metric can provide a numerical value regarding patients to be expected at the given unit. For example, scheduled surgeries can be used as part of metric for known patients that will likely be admitted to the healthcare facility for several days after their surgeries are complete. As another example, adverse weather forecasts can be used as part of metric for unknown patients that will likely be admitted to the healthcare facility due to an expected rise in flu or other infectious disease cases resulting from bad weather. Similarly, an infectious disease tracker can be used as part of the metric for unknown patients that will be admitted according to trends seen in infectious disease patterns in the area. Other metrics can include days of the week or special dates known to affect patient admission numbers, such as Thanksgiving, Christmas, and the Super Bowl. Still other metrics can include historical values and trends, such as the numbers of patients by unit on the same date in past years, as well as the increases or decreases in those numbers from the day before and to the day after in past years.

An Expected Value calculation for each metric 18 can take the metric calculations from Metric Calculation and Storage and assign confidence multipliers to those numerical values. Each confidence multiplier can have a value ranging from 0 to 1, which represents the certainty or likelihood that the numerical value will be accurate. For patients that are scheduled for surgeries, for example, the confidence multiplier for those patients will be relatively high, such as 0.95. Various factors can be used in assigning a confidence multiplier to a numerical value for a certain metric, such as the chance that a surgery is canceled or rescheduled, a patient does not show up, or a patient admission is not actually needed. In the case of scheduled surgeries, these likelihoods are low, such that the confidence multiplier can be relatively high. An example of a relatively low confidence multiplier might involve a metric for infectious disease tracking in the area, where the assigned confidence multiplier might be 0.60 due to the unpredictable nature of yet unknown patients and the possibility that newly sick people may not come in for treatment yet or may go to another facility.

Specific Metric selected for analysis and forecasting 20 can involve analyzing data and values from Metric Calculation and Storage 16 and Expected Value 18, which may include standard statistical methods, machine learning techniques, and/or deep learning methodologies. For example, the numerical values for the various metrics can be multiplied by their respective confidence multipliers to return an adjusted value for that metric. In a specific example, 20 patients may be scheduled for bariatric and joint replacement surgeries on a given date. The numerical value will then be 20 for the metric of scheduled surgeries. A confidence value of 0.95 can then be assigned to that metric, which returns an adjusted value of 19 patients expected to be admitted due to scheduled surgeries for that date. All of the various metric adjusted values can then be combined to result in a predicted patient census for a particular unit of the healthcare facility, and this process can be repeated for each unit of the healthcare facility. Further details and examples of data analysis and forecasting are provided below.

Stored Nurse to Patient Ratio relationships 24 can include data regarding the number of patients per nurse for each clinical unit of the healthcare facility. For example, there can be no more than 1 patient per nurse in an operating room, 2 patients per nurse for an intensive care unit (“ICU”), 4 patients per nurse in an emergency department, or 6 patients per nurse in a psychiatric ward. Of course, there are many other different types of units for healthcare facilities, and each different unit can have a separate nurse to patient ratio. These relationships can be mandated by state law and/or can be set by an individual healthcare facility to be stricter than state law where increased productivity standards may be desired. These ratio relationships can be used by Specific Metric analysis and forecasting 20 to help properly staff each unit. For example, where a predicted patient census for an ICU is 14 for a given shift, then the nurse staffing for that ICU shift should be at least 7 (i.e., maximum 2 ICU patients per ICU nurse).

Graphic representation of calculation outputs 28 can include generating a graphical format to display some or all of the relevant data and calculated values. This can include historical data, predicted patient censuses, scheduled staffing levels, and other data items of interest. Other data items and information that can be placed in a graphic representation are shown in and discussed with respect to FIG. 3A and other figures below involving a graphical user interface (“GUI”). After the graphic representation is generated, this can be pushed to a user computer having a user interface for display and interaction.

At the user computer, a user selects unit to analyze 30 using the user interface, which can involve selecting an ICU, an emergency department, an oncology unit, a pediatrics unit, or any other unit at the healthcare facility. Results are then displayed to the user at 32 for the selected unit. The displayed results can include the graphic generation of calculation outputs that were generated at 26, which may include data regarding the staffing level of the selected unit. A future shift for the unit can be selected as well, which can include a particular date and time, with displayed results then including the number of staff scheduled and the predicted patient census for the future shift.

An inquiry can then be made at 34 as to whether the staffing level of the selected unit is adequate. This can involve determining whether the number of nurses scheduled is greater than or equal to the number of nurses that are needed to work the future shift at the selected unit. If the staffing level is determined to be adequate at 34, then another unit can be selected for analysis. If the staffing level is determined not to be adequate at 34 and there is a shortfall in clinical labor, however, then automated healthcare staffing system 1 can automatically load balance to meet patient demands.

This can involve a subsequent inquiry being made at 38 as to whether internal staff is available. Internal staff can mean nurses that are employed by and regularly work at the healthcare facility. The inquiry can involve looking for nurses that meet the clinical inclusion criteria for working at the selected unit. For example, only nurses with specialized credentials may be allowed to work on a burn unit, such that only those credentialed nurses would be considered for the availability inquiry.

If qualified internal staff is available, then the internal staff can be assigned to the unit at 36. This can involve a notification being sent from the user computer to an outside device of the internal staff being assigned, such as, for example, a smart phone or other user mobile device. This assignment and notification process can be automatically performed by the system 1 if the staffing level for a unit is inadequate. In some arrangements, a manual option to confirm or reject the automated assignment may be available to the newly assigned staff.

If qualified internal staff is not available, or not enough qualified internal staff is available, then an alert can be sent to external contingent labor at 40. Contingent labor can include, for example, qualified nurses who are not employed by the healthcare facility but are rather employed by an external staffing agency and are on call for multiple healthcare facilities.

### Example Method

Turning next to FIG. 2A, a flowchart of an exemplary method 200 of providing a predicted patient census for a healthcare facility according to one embodiment of the present disclosure is provided. The predicted patient census can be for a future shift at the healthcare facility and can be an overall census or a census for an individual healthcare facility unit. After a start step 202, the computer system performing method 200 can receive statistical data from a plurality of data sources. These data sources can at least include a scheduling system, an ADT system, and an EMR system. Of course, further data sources can also be used, such as an ERP system and one or more external data sources.

At process step 206, the computer system can analyze the statistical data received, which may include standard statistical methods, machine learning techniques, and/or deep learning methodologies using preset preferences, parameters, and formulas. The analysis can include assigning numerical values for different metrics, such as scheduled surgeries, known upcoming procedures, weather data, infectious disease outbreak data for the surrounding area, and historical data related to the exact time, date, day of the week, and other factors, among many other possible metrics.

At process step 208, the computer system can forecast a patient census at the healthcare facility for a future shift or for the overall facility. This can be accomplished based on the analysis of statistical data received. Various metrics can be totaled according to one or more formulas to arrive at the predicted patient census. For example, a predicted patient census for a particular inpatient unit can include portions attributed to known surgeries and procedures, as well as portions attributed to unknown patients, such as possible flu victims or increases due to weather or other conditions.

At process step 210, the computer system can provide an output to a user. The output can include the predicted patient census, which may be presented in graphical format. The predicted patient census output can then be used staff the healthcare facility. For example, a predicted patient census for a future shift of a given unit might be 50. If there are not enough nurses scheduled for that future shift to cover 50 patients, then more nurses can be added. After providing the output to the user, method 200 then ends at end step 214.

Some of the steps of exemplary method 200 may be performed in different orders or in parallel. Also, the steps of exemplary method 200 may occur at two or more computers and/or servers, for example if the method is performed in a networked environment. Various steps may be optional.

Continuing with FIG. 2B, a flow chart illustrating additional steps that may be performed is provided. Method 201 includes the steps of method 200 and additionally includes further steps. An initial process step 203 can involve storing analysis parameters on the computer system. For example, a set of nurse to patient ratio relationships can be stored on the computer system as one form of analysis parameters. Each of the nurse to patient ratio relationships can reflect the maximum number of patients permitted per nurse for a unit of the healthcare facility as required by law, healthcare facility guidelines, or both. Other analysis parameters can also be stored on the computer system at step 203.

After an output is provided to a user at process step 210, a user request can be accepted at process step 211. In various embodiments, the computer system may present a user interface element for accepting user requests or other input. For example, a local computer may present the user interface element by calling a function to cause the display of a user interface element. On the other hand, a remote computer may present a user interface element to the user by sending a signal that causes a local computer to display such a user interface element. Alternatively, the remote computer may present a user interface element to the user by sending a document to a local computer for display that comprises such a user interface element. The user interface element may be configured to receive button selections, pull down menu selections, text entries, or other forms of user input. In various embodiments, the user request can relate to information for a future shift at a unit of the healthcare facility.

At process step 212, information is provided to the user, which can be in response to the user request. For example, the information can include the number of nurses scheduled for a future shift at a healthcare facility unit, as well as the predicted patient census for that shift based on the analyzed statistical data. In various embodiments, the information can include scheduled nurses and predicted patient censuses for multiple shifts and multiple units. The provided information can also include historical data, which can be presented in graphical format so that trends can be easily spotted.

At process step 213, the computer system can automatically dynamically update the predicted patient census as the future date approaches. This can essentially involve repeating steps 204-208, which can occur whenever new statistical data is received. For example, the ADT system can record a single new patient arriving at the emergency department with second degree burns, which can result in one new known patient having a 0.95 confidence multiplier of being admitted to a burn unit the next day. This new data item of 0.95 more patients can be added to the metrics for the burn unit as part of the dynamic update to the predicted patient census for all shifts for the burn unit for the next three days.

Some of the steps of exemplary method 201 may be performed in different orders or in parallel. Also, the steps of exemplary method 201 may occur at two or more computers and/or servers, for example if the method is performed in a networked environment. As one possible example, steps 210, 211, and 212 may occur on a local computer, while steps 203, 204, 206, 208, and 213 may occur on a remote computer. In such an example, the local computer may be providing an interface for accessing and saving data or files on a storage device connected to a remote server. Various steps may be optional.

### Example Displays and User Interfaces

Transitioning to FIG. 3A, an example screenshot of information for an automated healthcare staffing system is shown. Screenshot 300 can be formatted for display on a desktop, laptop, thin client monitor coupled to a remote server, or other similar computing device, and can be designed for use by a staffer or other manager of a healthcare facility. Alternatively, a streamlined version of screenshot 300 can be presented on a smart phone or other suitable mobile device. Screenshot 300 can include the graphic representation of calculation outputs 26 and results displayed to user 32 of FIG. 1C. Screenshot 300 can present informational items as well as several GUI elements that allow for user input, such as the selectable items of menu 301 for “Example Hospital.” In some arrangements, the informational items shown in screenshot 300 can be historical. In other arrangements, these items can be forecasted items for future dates or shifts. A combination of historical and forecasted data can also be presented.

Summary values 302 can represent the percentages and numbers for different items on a selected date, such as, for example, the percentage of shifts optimally staffed, the longest streak of shifts optimally staffed, and the percentage of shifts filled by internal staff, among other types of summary values. These values can be presented for the overall healthcare facility or can be broken down by unit.

X-Y graph 303 can present data on various possible items. For example, a day to day presentation of optimal vs. actual care capacity can chart how closely actual staffing measured up against optimal staffing for past dates and can forecast these values for future dates based on current schedules and current predicted patient censuses. For the highlighted date of Thursday March 3, for example, the optimal number of nurses for the healthcare facility would have been 91, while the actual number of nurses that worked that date was 89.

Bar graph 304 can present data on staffing mix by unit for a given date or shift. Each bar can represent a percentage of the actual staffing for a unit of the healthcare facility, and the components of the bar can include percentages for unit staff, float nurses from other units within the healthcare facility, and nurses from an external staffing agency. As will be appreciated, costs increase and efficiencies decrease when external nurses are used, such that staffers and other management personnel will aim to minimize the percentage of staffing attributed to external nurses.

Pie graph 305 can present data on overall staffing mix for the entire healthcare facility. One category can be for internal nurses, while another can be for external nurses provided by an outside staffing agency. Again, operators may tend to increase the percentage of internal nurses staffed over time for reasons of cost and efficiency. In various embodiments, system alerts may be provided when the percentage of nurses from external sources grows to be too large.

FIG. 3B illustrates an example graphical user interface for an automated healthcare staffing system. It will be understood that the GUI of FIG. 3B and all other GUIs depicted herein are exemplary and should not be viewed as limiting. Similar to screenshot 300, GUI 310 can be designed for use by a staffer or other management personnel. GUI 310 can be a staffing page that may appear when certain selections are made on the interface shown in screenshot 300 above. For example, GUI 310 may appear as a result of making selections on the “Schedules” button within the menu 301 of screenshot 300. The information presented may relate to the exact personnel staffed for a given shift on a specific unit.

GUI 310 can include a menu 311 having various user selectable items, which can help to navigate a user forward or backward within the system. An “Actual Staffed” panel 312 can display all the nurses that are currently scheduled for the selected future shift. As shown, this can include the name, title, and a thumbnail picture for the scheduled nurse. In some embodiments, each displayed nurse can include a link that provides further information on that individual when clicked, such as qualifications and other scheduled shifts, for example.

A “Call Outs” panel 313 can display the nurses that were scheduled for the selected future shift but have called in sick or otherwise had their shift canceled. Information here can also include the name, title, and thumbnail picture for the nurse, as well as the time of and reason for the call in or cancellation for that nurse. Each displayed nurse can include a link that provides further information, and additional details can be added by the user, such as to a digital notepad or line item for each call out.

An “Available Fill Ins” panel 314 can display the nurses that may be available to fill in for the selected shift. These available nurses can be categorized as unit staff or external nurses. Another category may also be included for possible float nurses employed by the healthcare facility but staffed for at different unit or as permanent float nurses. As shown, panel 314 can include buttons to broadcast that the selected shift is available. One button can broadcast to all available unit staff, while another button can broadcast to external nurses from a staffing agency. In some embodiments, these buttons may be manually selected by a user to broadcast to available nurses. Alternatively, or in addition, some embodiments may involve the system automatically broadcasting to one or both sets of nurses, such as when there is an urgent need for added staffing for a shortly upcoming shift.

Moving to FIGS. 3C-3F, various mobile device GUIs are provided for users of an automated healthcare staffing system. FIG. 3C illustrates an example graphical user interface for internal staff of the system. GUI 350 can include a calendar 351 having multiple dates that may be selected by a user. A date or shift summary 352 can be presented beneath the calendar 351 for a selected date or shift, with this summary including the day relative to the current day, a selected shift and its start and end times, and other informational items. A compact list 353 of the nurses or other staff scheduled for the selected shift can be presented proximate the shift summary 352. This compact list 353 may be scrolled to the right or left in the event that all scheduled staff cannot fit in the space available on the GUI 350. A panel of user selections 354 can allow a user to choose different options, such as a call out button and links to and from the schedule, notifications, shift swaps, available staff, and settings, among other possible selections.

FIG. 3D illustrates another example graphical user interface for internal staff of an automated healthcare staffing system. GUI 360 can represent a page that appears when a user selects the call out button of GUI 350. A call out button can be selected, for example, when an internal staff member is calling in sick or otherwise canceling his or her scheduled shift. A lead informational panel 361 can provide information regarding the date, shift, and unit for which the internal staff member is scheduled. This can often be a shift that is the same day or next day. A secondary informational panel 362 can let the user know whether the shift is adequately staffed or is already short on personnel. Another informational panel 363 can inform the user how many call outs he or she already has for a given period of time, such as the current pay period, as well as the maximum number of call outs allowed per facility policy. A call out button 364 can be pressed to confirm that the internal staff member is calling out of his or her shift.

FIG. 3E illustrates an example graphical user interface for external staff of the system. External staff can be nurses or other personnel that are not employed by a healthcare facility, but rather are on call in the event that any of a number of participating facilities contract out for additional staff from an outside source, such as an external staffing agency. GUI 370 can present one or more alerts to contingent labor when external staffing is being requested, such as at item 40 of FIG. 1C. A header 371 can list the types of assignments being requested, such as contract or per diem assignments. Specific assignment alerts can be presented in assignment list 372. Each assignment alert can include the date, shift, healthcare facility, and type of assignment, among other pertinent informational items. Although only one assignment is shown in assignment list 372, it will be understood that multiple available assignment alerts may be presented at a given time. The assignment list 372 may be scrolled up or down in the event that there are more assignments than can fit on GUI 370 at one time. A panel of user selections 373 can allow a user to request a specific available shift, chat with an administrator or staffer offering the shift, or close the GUI 370, among other possible options.

FIG. 3F illustrates another example graphical user interface for external staff of an automated healthcare staffing system. GUI 380 can represent an assignment details page that appears when a user selects a specific assignment listed in GUI 370. A map 381 can display where the healthcare facility providing the assignment is located. An informational panel 382 can provide added details regarding the assignment, such as the unit or department, the amount of pay for the shift, the date and time of the shift, and the certifications that are required to be able to qualify for the shift. A panel of user selections 383 can allow a user to request that detailed shift, chat with an administrator or staffer offering the shift, or close the GUI 380, among other possible options. While various screenshot displays and GUIs have been provided for purposes of illustration, it will be appreciated that many other informational displays and GUIs may be used for different users in the disclosed automated healthcare staffing systems.

### Example Implementations of Automated Staffing

Various ways of providing automated staffing for a healthcare facility will now be discussed. As an initial matter, certain aspects of automatically analyzing received statistical data are highlighted. FIG. 4 illustrates a flowchart of example details for analyzing statistical data in order to provide staffing for a healthcare organization. In particular, FIG. 4 corresponds to the analyzing statistical data step 206 of method 201 in FIG. 2B. The functions for this step can be performed by one or more modules or programs, such as Data Analysis 154 in FIG. 1B.

At step 206A, the computer system can assign numerical values to statistical data that is received from different data sources. Various items in the statistical data can be used for different metrics, and each metric can be assigned a numerical value regarding the number of patients to be expected at a unit of the healthcare facility. Metrics can include the number of known patients to be expected, such as patients scheduled for surgeries or procedures, as well as expected transfers from emergency department or ICU admissions. Metrics can also include unknown patients to be anticipated based on factors such as historical data for past unit patient censuses. These can include overall patient volumes for different shifts on a given date, day of the week, and time of the year, as well as increases or decreases in the patient volume for the shifts before and after the analyzed shift. Metrics used to predict the number of unknown patients that will be admitted can also include weather conditions, infectious disease outbreaks in the area, and other relevant conditions.

At step 206B, the computer system can assign confidence multipliers to the numerical values given to the different metrics. Each confidence multiplier can reflect the likelihood of that metric reaching its full value, and as such can vary from 0 to 1, with 1 being 100% certainty. Various factors can be considered in assigning confidence multipliers. For example, the confidence multiplier for patients due to an infectious disease outbreak may be low at the start of an outbreak but increase over time as more people develop symptoms and come in for treatment. As another example, the confidence multiplier for a person arriving at the emergency department by ambulance might be 0.90, as this is the likelihood that this person will be admitted as inpatient after their emergency procedures are done.

At step 206C, the computer system can use the numerical values and confidence multipliers in at least one statistical model to compute a plurality of outputs relating to a predicted patient census for one or more units of the healthcare facility. Different statistical models can be used for different units of the healthcare facility. For example, a predicted patient census for an emergency department can result from a seasonal autoregressive integrated moving average (“SARIMA”) statistical model applied to the numerical values and confidence multipliers derived from the statistical data relating to emergency department admissions. A SARIMA model and/or other statistical models can similarly be used with the received data to predict patient censuses for other healthcare facility units.

As will be readily appreciated, the data for various units across a healthcare facility can be interrelated and somewhat dependent. For example, once a patient census is known or even predicted for an emergency department, then further statistical models can be applied to predict at least portions of a patient census for other units. This is due to the typically high transfer rates from an emergency department to other units of the healthcare facility. Individual patient records and characteristics can be analyzed using one or more statistical models, such as a naïve Bayes conditional probability, generalized linear egression with a logit link function (logit-linear regression), Multivariate adaptive regression splines (“MARS”), Regression random forests, and SARIMA models.

These models can then be used to predict patient admissions to other units from known patients in the emergency department. The exact unit that a given patient would be transferred to will depend upon the reason for admission to the emergency department and any other known factors. For example, a burn victim would most likely be admitted to a burn unit, while a sick child would most likely be admitted to a pediatric unit from the emergency department. Further statistical models can then be used to predict the lengths of stays for inpatient admissions from an emergency department, which can then be used to predict the number of inpatient discharges from the healthcare facility for a given date. These predictions can then be used to help calculate predicted patient censuses for future shifts based on the expected discharge dates and times for inpatient admissions from the emergency departments.

As a simplified non-limiting example, data can be received from a plurality of different data sources, and a sample Bayesian calculation by the system for a given patient can take the form of:

$$P ( A \vert B ) = \frac{P ( B \vert A ) P ( A )}{P ( B )}$$

Where patient data items can include, for example, Emergency Severity Index Designation Level 1-5, Arrival Mode, Primary Complaint, Age, Sex, Blood Pressure, Respiratory Rate, PulseOx, and the like. The system can run these specific calculations for each patient separately and provide updates with new and live data. Forecasts of inpatient census are then aggregated and determined to each level of care type/unit based on the numbers calculated for each known patient.

As an illustrative non-limiting example of 7 patients admitted to an emergency department during a given shift, a Bayesian calculation for each of the patients might produce the following results:

P₁=0.87, P₂=0.43, P₃=0.89, P₄=0.94, P₅=0.67, P₆=0.77, P₇=0.88

where Pₓ is the probability for each of the patients being admitted as inpatient to the healthcare facility from the emergency department. Adding up all of these results can then provide a component of a predicted patient census that includes these 7 patients. That is, the predicted number of patients admitted as inpatient to the healthcare facility from this component of 7 emergency department patients will be 5.45. Other components having different metrics for other known and unknown patients can also be combined to give a total predicted patient census. Of course, different amounts of patients and different probability results can exist in other situations.

FIG. 5 illustrates a flowchart of an example overview method 500 of providing automated staffing for a healthcare organization. After a start step 502, the computer system performing method 500 select a healthcare department or unit at process step 504. This selection can be performed automatically, or can be in response to a user input, such as the user selection 30 in FIG. 1C.

At process step 506, the computer system can convert a predicted patient census for a future shift to a staffing need. This can involve already having a predicted patient census for the selected unit, with details for acquiring this item set forth in the methods of FIGS. 2A, 2B, and 4 above. Converting the predicted patient census to a staffing need can include using a stored nurse to patient ratio for that unit to determine the number of nurses needed for the predicted patient census for the future shift. For example, if the predicted patient census for an ICU is 20 for the next two shifts and the nurse to patient ratio is 1:2, then the staffing need is 10 nurses for both of the next two shifts.

At process step 508, the computer system can compare the staffing need to the staffing schedule for the selected department or unit. Continuing with the previous example, the staffing schedule for the ICU for the next shift might be 12 nurses, while the staffing schedule for the subsequent shift might be 8 nurses due to call outs and cancellations for that shift.

An inquiry can then be made at decision step 510 as to whether the staffing need is greater than the number of nurses scheduled for a given shift. In the case of the first shift, there are 12 nurses actually scheduled and a need of only 10 nurses based on the predicted patient census. In this case the negative answer to the inquiry results in the method skipping to decision step 516.

In the case of the next shift, however, there are 8 nurses scheduled when there is a need for 10. Accordingly, the method then proceeds to process step 512 where the computer system provides a list of available staff is for the future shorthanded shift. This list can include internal nurses employed by the healthcare facility as well as external nurses available through a staffing agency.

At process step 514 the computer system can procure additional staff for the future shift. This can be done automatically, such as where internal staff on the available list is automatically assigned to the unit for that shift (e.g., item 36 in FIG. 1C). Alternatively, this can be done by way of manual input from a user, such as by a specific nurse selection or contact. It will be understood that alerts may be automatically generated and sent to both internal and external staff when there is a staffing need for a shift, and that both internal and external staff may be automatically assigned to a shift by the system. Similarly, these steps may be performed manually by a user if desired.

At decision step 516, an inquiry is made as to whether all desired departments or units have been considered. If so, then the method ends at end step 518. If not, then the method reverts to step 504 and all steps can be repeated for a different unit and/or different shift. Some of the steps of method 500 may be performed in different orders or in parallel. Also, the steps of method 500 may occur in two or more computers, for example if the method is performed in a networked environment. Various steps are optional, and various additional steps may be added.

For example, one or more steps may be added to account for possible or likely “holes” in a staffing schedule. Holes in a staffing schedule can refer to nurses who are scheduled but who call out or otherwise cancel their scheduled shift. The likelihood of such staffing holes can increase for certain nurses and for certain dates or other circumstances. For example, it is generally well known that a higher percentage of nurses will call out from their scheduled shifts on Thanksgiving, Christmas, and other certain days of the year. Weather conditions, traffic, and other circumstances may also factor into the possibility or likelihood of holes occurring in an otherwise well-staffed shift schedule.

To account for the possibility of staffing holes, extra steps can include calculating a predicted nurse census for a given shift, and then adding extra nurses to the schedule to account for the predicted number of absences. For example, where a scheduled shift may include 15 nurses for a given unit, a predicted nurse census may be 13 or 14 nurses based on the likelihood of one or two call outs for that shift. One or two extra nurses may be accordingly scheduled for that shift. Alternatively, so as to avoid overstaffing if all scheduled nurses report for the shift, a preliminary stand-by or contingent alert may be sent to available internal and/or external nurses.

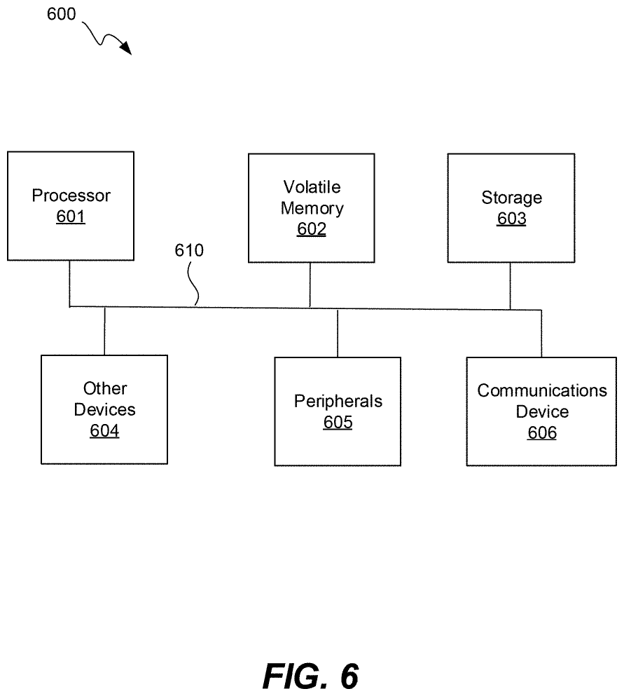

FIG. 6 illustrates a diagram of an example computer that may perform processing for various embodiments and components of the present disclosure. Computer 600 may perform operations consistent with some embodiments. The architecture of computer 600 is exemplary. Computers can be implemented in a variety of other ways. A wide variety of computers can be used in accordance with the embodiments herein.

Processor 601 may perform computing functions such as running computer programs. The volatile memory 602 may provide temporary storage of data for the processor 601. RAM is one kind of volatile memory. Volatile memory typically requires power to maintain its stored information. Storage 603 provides computer storage for data, instructions, and/or arbitrary information. Non-volatile memory, which can preserve data even when not powered and including disks and flash memory, is an example of storage. Storage 603 may be organized as a file system, database, or in other ways. Data, instructions, and information may be loaded from storage 603 into volatile memory 602 for processing by the processor 601.

The computer 600 may include peripherals 605. Peripherals 605 may include input peripherals such as a keyboard, mouse, trackball, video camera, microphone, and other input devices. Peripherals 605 may also include output devices such as a display. Peripherals 605 may include removable media devices such as CD-R and DVD-R recorders/players. Communications device 606 may connect the computer 100 to an external medium. For example, communications device 606 may take the form of a network adapter that provides communications to a network. A computer 600 may also include a variety of other devices 604. The various components of the computer 600 may be connected by a connection medium 610 such as a bus, crossbar, or network.

Although the foregoing disclosure has been described in detail by way of illustration and example for purposes of clarity and understanding, it will be recognized that the above described disclosure may be embodied in numerous other specific variations and embodiments without departing from the spirit or essential characteristics of the disclosure. Certain changes and modifications may be practiced, and it is understood that the disclosure is not to be limited by the foregoing details, but rather is to be defined by the scope of the appended claims.

What is claimed is:

1. A method for staffing a healthcare facility, the method performed by a computer system, the method comprising:
storing a set of nurse to patient ratio relationships on the computer system, wherein each of the nurse to patient ratio relationships reflect the maximum number of patients permitted per nurse for a unit of the healthcare facility;
receiving at the computer system statistical data from a plurality of data sources, wherein the plurality of data sources includes at least a scheduling system, an admission discharge transfer system, and an electronic medical record system;
calculating numerical values for a plurality of metrics based on items within the statistical data, wherein each of the plurality of metrics relate to a patient census within the healthcare facility;
assigning confidence multipliers to each of the metrics having a numerical value, wherein each confidence multiplier ranges from 0 to 1;
using the numerical values and confidence multipliers in at least one statistical model to compute a plurality of outputs, wherein each output relates to a predicted patient census for a unit of the healthcare facility, and wherein the outputs include at least a predicted patient census for an emergency department, a predicted number of patients admitted as inpatient to the healthcare facility from the emergency department, and a predicted number of inpatient discharges from the healthcare facility;
saving the outputs on the computer system;
generating a graphical representation of the outputs;
displaying the graphical representation of the outputs to a user of the computer system;
accepting at the computer system a request from the user to analyze a selected unit of the healthcare facility, wherein the computer system includes a user interface element configured to receive user input;
providing information to the user regarding the selected unit, wherein the information comprises the number of nurses scheduled to work a future shift at the selected unit and the number of nurses that are needed to work the future shift based on a predicted patient census for the selected unit;
determining whether the number of nurses scheduled is greater than or equal to the number of nurses that are needed to work the future shift at the selected unit;
determining whether any internal nurses are available when the number of nurses that are needed is greater than the number of nurses that are scheduled to work the future shift at the selected unit, wherein the internal nurses comprise nurses employed by the healthcare facility;
assigning one or more internal nurses to work the future shift at the selected unit when the number of nurses that are needed is greater than the number of nurses that are scheduled to work the future shift at the selected unit and internal nurses are available; and
sending an alert to nurses employed by an outside staffing agency when the number of nurses that are needed is greater than the number of nurses that are scheduled to work the future shift at the selected unit and internal nurses are not available.

2. The method of claim 1, further comprising:
providing to the user a list of available nurses when the number of nurses that are needed is greater than the number of nurses that are scheduled to work the future shift at the selected unit.

3. The method of claim 2, wherein the list of available nurses includes internal nurses and nurses employed by an external staffing agency.

4. The method of claim 1, further comprising:
converting a predicted patient census for the selected unit to the number of nurses that are needed to work the future shift based on a stored nurse to patient ratio relationship for the selected unit.

5. The method of claim 1, further comprising:
dynamically updating the predicted patient census for the selected unit as the future shift approaches.

6. The method of claim 1, wherein the at least one statistical model includes a seasonal autoregressive integrated moving average statistical model.

7. The method of claim 1, wherein the plurality of data sources further includes a weather application, a healthcare news application, or an infectious disease tracker.

8. The method of claim 1, wherein the graphical representation includes a combination of historical and forecasted patient census information.

9. The method of claim 1, wherein the information provided to the user regarding the selected unit includes nurses currently scheduled for the future shift and nurses who have called out of the future shift.

10. The method of claim 1, further comprising:
calculating a predicted nurse census for the future shift at the selected unit; and
increasing the number of nurses scheduled to work the future shift when the number of nurses that are needed is greater than the predicted nurse census.

11. A non-transitory computer-readable medium containing instructions for staffing a healthcare facility, the instructions for execution by a computer system, the non-transitory computer-readable medium comprising:
instructions for storing a set of nurse to patient ratio relationships on the computer system, wherein each of the nurse to patient ratio relationships reflect the maximum number of patients permitted per nurse for a unit of the healthcare facility;
instructions for receiving at the computer system statistical data from a plurality of data sources, wherein the plurality of data sources includes at least a scheduling system, an admission discharge transfer system, and an electronic medical record system;
instructions for calculating numerical values for a plurality of metrics based on items within the statistical data, wherein each of the plurality of metrics relate to a patient census within the healthcare facility;
instructions for assigning confidence multipliers to each of the metrics having a numerical value, wherein each confidence multiplier ranges from 0 to 1;
instructions for using the numerical values and confidence multipliers in at least one statistical model to compute a plurality of outputs, wherein each output relates to a predicted patient census for a unit of the healthcare facility, and wherein the outputs include at least a predicted patient census for an emergency department, a predicted number of patients admitted as inpatient to the healthcare facility from the emergency department, and a predicted number of inpatient discharges from the healthcare facility;
instructions for saving the outputs on the computer system;
instructions for generating a graphical representation of the outputs;
instructions for displaying the graphical representation of the outputs to a user of the computer system;
instructions for accepting at the computer system a request from the user to analyze a selected unit of the healthcare facility, wherein the computer system includes a user interface element configured to receive user input;
instructions for providing information to the user regarding the selected unit, wherein the information comprises the number of nurses scheduled to work a future shift at the selected unit and the number of nurses that are needed to work the future shift based on a predicted patient census for the selected unit;
instructions for determining whether the number of nurses scheduled is greater than or equal to the number of nurses that are needed to work the future shift at the selected unit;
instructions for determining whether any internal nurses are available when the number of nurses that are needed is greater than the number of nurses that are scheduled to work the future shift at the selected unit, wherein the internal nurses comprise nurses employed by the healthcare facility;
instructions for assigning one or more internal nurses to work the future shift at the selected unit when the number of nurses that are needed is greater than the number of nurses that are scheduled to work the future shift at the selected unit and internal nurses are available; and
instructions for sending an alert to nurses employed by an outside staffing agency when the number of nurses that are needed is greater than the number of nurses that are scheduled to work the future shift at the selected unit and internal nurses are not available.

12. The non-transitory computer-readable medium of claim 11, further comprising:
instructions for providing to the user a list of available nurses when the number of nurses that are needed is greater than the number of nurses that are scheduled to work the future shift at the selected unit.

13. The non-transitory computer-readable medium of claim 11, further comprising:
instructions for dynamically updating the predicted patient census for the selected unit as the future shift approaches.

14. The non-transitory computer-readable medium of claim 11, wherein the at least one statistical model includes a seasonal autoregressive integrated moving average statistical model.

15. The non-transitory computer-readable medium of claim 11, wherein the plurality of data sources further includes a weather application, a healthcare news application, or an infectious disease tracker.

16. The non-transitory computer-readable medium of claim 11, further comprising:
instructions for calculating a predicted nurse census for the future shift at the selected unit; and
instructions for increasing the number of nurses scheduled to work the future shift when the number of nurses that are needed is greater than the predicted nurse census.

17. An automated healthcare staffing system, comprising:
at least one memory that contains non-transitory processor-executable instructions; and
a processor coupled to the at least one memory, the processor being configured to execute the processor-executable instructions, wherein the processor-executable instructions include:
instructions for storing a set of nurse to patient ratio relationships on the computer system, wherein each of the nurse to patient ratio relationships reflect the maximum number of patients permitted per nurse for a unit of the healthcare facility;
instructions for receiving at the computer system statistical data from a plurality of data sources, wherein the plurality of data sources includes at least a scheduling system, an admission discharge transfer system, and an electronic medical record system;
instructions for calculating numerical values for a plurality of metrics based on items within the statistical data, wherein each of the plurality of metrics relate to a patient census within the healthcare facility;
instructions for assigning confidence multipliers to each of the metrics having a numerical value, wherein each confidence multiplier ranges from 0 to 1;
instructions for using the numerical values and confidence multipliers in at least one statistical model to compute a plurality of outputs, wherein each output relates to a predicted patient census for a unit of the healthcare facility, and wherein the outputs include at least a predicted patient census for an emergency department, a predicted number of patients admitted as inpatient to the healthcare facility from the emergency department, and a predicted number of inpatient discharges from the healthcare facility;
instructions for saving the outputs on the computer system;
instructions for generating a graphical representation of the outputs;
instructions for displaying the graphical representation of the outputs to a user of the computer system;
instructions for accepting at the computer system a request from the user to analyze a selected unit of the healthcare facility, wherein the computer system includes a user interface element configured to receive user input;
instructions for providing information to the user regarding the selected unit, wherein the information comprises the number of nurses scheduled to work a future shift at the selected unit and the number of nurses that are needed to work the future shift based on a predicted patient census for the selected unit;
instructions for determining whether the number of nurses scheduled is greater than or equal to the number of nurses that are needed to work the future shift at the selected unit;
instructions for determining whether any internal nurses are available when the number of nurses that are needed is greater than the number of nurses that are scheduled to work the future shift at the selected unit, wherein the internal nurses comprise nurses employed by the healthcare facility;
instructions for assigning one or more internal nurses to work the future shift at the selected unit when the number of nurses that are needed is greater than the number of nurses that are scheduled to work the future shift at the selected unit and internal nurses are available; and
instructions for sending an alert to nurses employed by an outside staffing agency when the number of nurses that are needed is greater than the number of nurses that are scheduled to work the future shift at the selected unit and internal nurses are not available.

18. The system of claim 17, wherein the processor-executable instructions further include:
instructions for providing to the user a list of available nurses when the number of nurses that are needed is greater than the number of nurses that are scheduled to work the future shift at the selected unit.

19. The system of claim 17, wherein the processor-executable instructions further include:
instructions for dynamically updating the predicted patient census for the selected unit as the future shift approaches.

20. The system of claim 17, wherein the at least one statistical model includes a seasonal autoregressive integrated moving average statistical model.
